(19) United States
(12) Patent Application Publication
(10) Pub. No.: US 2010/0132098 A1
(43) Pub. Date: Jun. 3, 2010

(54) Swimming Goggle Structure

(75) Inventor: Chih-Cheng Shiue, Taipei (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: QBAS CO., LTD., Taipei (TW)

(21) Appl. No.: 12/630,205

(22) Filed: Dec. 3, 2009

(30) Foreign Application Priority Data
Dec. 3, 2008 (TW) 97146912

(51) Int. Cl. A61F 9/02

(52) U.S. Cl. 2/431

(57) ABSTRACT

A swimming goggle structure is provided. The swimming goggle structure comprises a frame assembly and a glass assembly. The frame assembly defines a receiving space and comprises a slot so the glass assembly is adapted to be embedded in the slot. The glass assembly comprises a connecting device and a functional lens. The connecting device extends from the frame assembly outward to a user's eyes. The connecting device has an outer end and an inner end. The outer end is disposed in the slot, while the inner end is connected to the functional lens. The distance between the user's eyes and the outer end of the connecting device is greater than the distance between the user's eyes and the inner end of the connecting device.

This application claims priority to Taiwan Patent Application No. 097146912 filed on Dec. 3, 2008.

### CROSS-REFERENCES TO RELATED APPLICATIONS

Not applicable.

### BACKGROUND OF THE INVENTION

1. Field of the Invention

The present invention relates to a swimming goggle structure, and more particularly, to a swimming goggle structure having a stereoscopic functional lens.

2. Descriptions of the Related Art Swimming goggles are often used in various snorkeling sports to prevent the surrounding water from seeping into and irritating the user's eyes. Thereby, a comfortable environment can be maintained for the user to participate in a snorkeling sport successfully.

In reference to both FIGS. 1 and 2, a conventional swimming goggle structure 1 is shown therein. The conventional swimming goggle structure 1 comprises a frame assembly 11, a mask 13 and at least one lens 122. The lens 122 is received in a receiving space formed by the frame assembly 11 and connects with the frame assembly 11 directly. The mask 13, which covers the frame assembly 11 and the lens 122, is provided for the user to wear around the face.

When the swimming goggle structure 1 is used by the user, the mask 13 is restrained in outline by the profile of the user's face and, especially, the mask 13 must be sealed against the user's nose completely to ensure safety in underwater sports. Because the mask 13 is restrained in outline, there is a significant distance between the user's face and the frame assembly 11 and the lens 122 joined with the mask 13. As shown in FIG. 2, a distance of more than 16 cm is usually kept between the lens 122 of the swimming goggle structure 1 and the user's eyes to provide enough space for the mask 13 to fit entirely against critical sites on the face such as the eyes and the nose.

As described above, a distance of more than 16 cm is often needed between the lens 122 and the user's eyes. As a result, the lens 122 of the conventional swimming goggle structure 1 must be correspondingly made to have a certain area to provide the user with an optimum field of vision. For example, lenses of typical conventional swimming goggles are mostly much larger than myopic lenses or hyperopic lenses for use on land. Otherwise, if the lens 122 of the swimming goggle has too small of an area, the user would have a narrow field of vision or even tunnel vision, which is unfavorable for safety in underwater sports.

Because the lens of the conventional swimming goggle has an area much larger than that of common lens for use on land, a large lens must be used in fabricating the swimming goggle. Consequently, the manufacturing process is both time consuming and labor consuming, leading to high costs. Furthermore, large lenses exhibit poor shock resistance. When myopic lenses or hyperopic lenses are to be fabricated, the materials and area necessary for the lenses as well as the production time are several times larger than common lenses for use on land. In view of this, it is important to provide a swimming goggle structure that can be made with less production time, fewer materials and lower costs.

### SUMMARY OF THE INVENTION

One objective of the present invention is to provide a swimming goggle structure. A lens used in the swimming goggle structure is closer to the user's eyes as compared to the conventional swimming goggle structure. Therefore, a large field of vision can be obtained by using of a small lens and the problem of tunnel vision can be obviated.

The swimming goggle structure disclosed in the present invention comprises a frame assembly and a lens assembly. The frame assembly defines a receiving space and has a slot formed therein. The lens assembly comprises a connecting device and a functional lens. The connecting device extends from the slot toward a user's eyes, and has an outer end and an inner end. The outer end is adapted to fit in the slot and the inner end opposite to the outer end joins with the functional lens. The distance between the outer end of the connecting device and the user's eyes is greater than the distance between the inner end of the connecting device and the user's eyes. Consequently, the functional lens is closer to the user's eyes, so a lens with a smaller area can be used without affecting the normal vision of the user, thereby saving the labor and cost necessary for producing the lens and strengthening the shock resistance of the lens.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic view of a conventional swimming goggle structure;

FIG. 2 is a top view of the conventional swimming goggle structure;

FIG. 3A is a schematic view of a swimming goggle structure of the present invention;

FIG. 3B is a schematic view of the swimming goggle structure of the present invention;

FIG. 3C is a schematic view of the swimming goggle structure of the present invention;

FIG. 4 is a top view of the swimming goggle structure of the present invention;

FIG. 5 illustrates the distance between the functional lens of the swimming goggle structure of the present invention and the user's eyes;

FIG. 6 is a schematic view illustrating the connection between a transparent material and the functional lens in the swimming goggle structure of the present invention;

FIG. 7A is a schematic view illustrating the relationships between the swimming goggle structure of the present invention and the user's eyes; and

FIG. 7B is a schematic view illustrating the relationships between the swimming goggle structure of the present invention and the user's eyes.

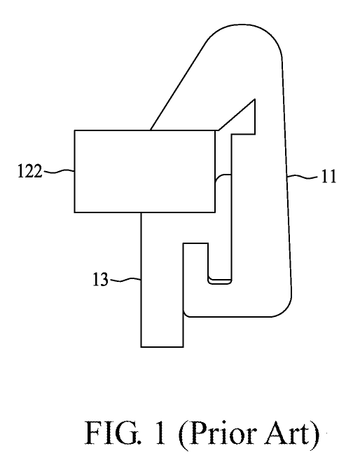

### DESCRIPTION OF THE PREFERRED EMBODIMENT

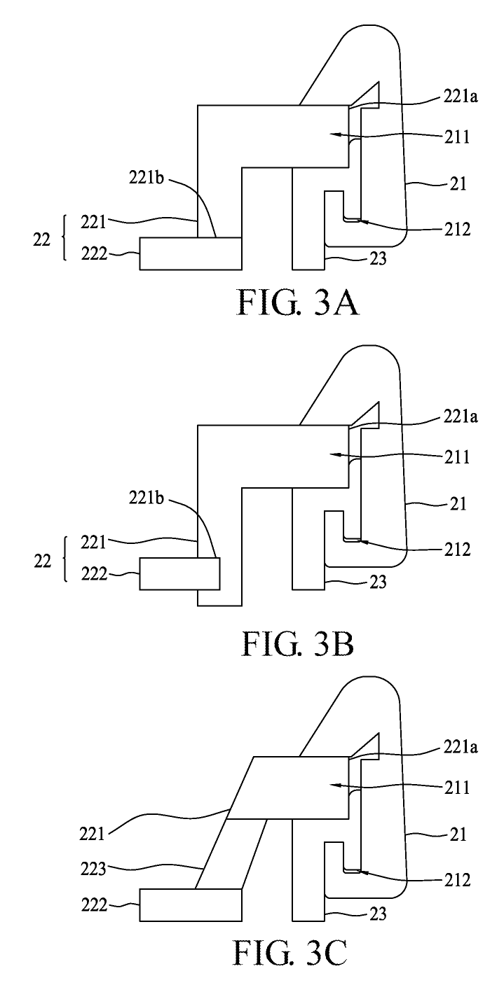

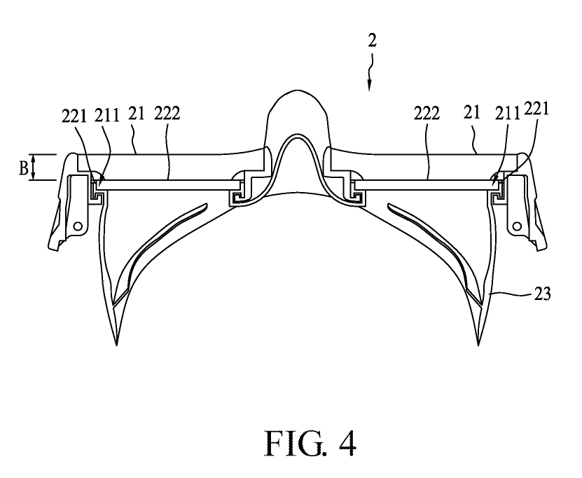

As shown in FIGS. 3A, 3B, 3C and 4, a swimming goggle structure 2 of the present invention comprises a frame assembly 21, a lens assembly 22 and a mask 23. The frame assembly 21 defines a receiving space 211 and is formed with a slot 212 around the receiving space 211. The mask 23 is adapted to cover and connect with the frame assembly 21 and the lens assembly 22 for the user to wear to protect the user's eyes and nose. The mask 23 further connects to a fastening device (not shown), the fastening device can be, but not limited, a belt, and the fastening device is used for the swimming structure 2 enclosing onto the user. Furthermore, the mask 23 comprises a skirt portion (not shown), and the swimming goggle structure 2 is adapted to attach onto the user's face tight by the skirt portion preventing water from permeating into the user's eyes and nose. The present invention is characterized in that the lens assembly 22 comprises a connecting device 221 and a functional lens 222 adapted to decrease the distance between the lens and the user's eyes and, therefore, the area of the lens. Both the connecting device 221 and the functional lens 222 are made of a transparent material, which is to be detailed later.

In particular, the connecting device 221 comprises an outer end 221a and an inner end 221b. The outer end 221a fits in the slot 212 of the frame assembly 21 so that the lens assembly 22 is adapted to be embedded in the receiving space 211. On the other hand, the inner end 221b of the connecting device 221 connects to the functional lens 222. The distance between the outer end 221a of the connecting device 221 and the user's eyes is greater than the distance between the inner end 221b of the connecting device 221 and the user's eyes; that is, the lens assembly 22 of the present invention is characterized in that the connecting device 221 and the lens 222 join together to form a stereoscopic lens, with the lens 222 located in the central area closer to the user's eyes than the connecting device 221 located in the periphery. The connecting device 221 is primarily made of a composite material including tempered glass, transparent silica gel, transparent plastics or transparent acrylics. The functional lens 222 is made of a glass material, which is identical to that of conventional lenses and thus will not be further described herein.

FIGS. 3A, 3B and 3C are schematic views illustrating how the connecting device 221 connects with the functional lens 222. In FIG. 3A, the inner end 221b of the connecting device 221 connects to the functional lens 222 by an adhesive and extends from the frame assembly 21 toward the functional lens 222 so that the functional lens 222 is closer to the user's eyes. In FIG. 3B, the inner end 221b of the connecting device 221 connects to the functional lens 222 integrally through an injection molding process and extends from the frame assembly 21 toward the functional lens 222 so that the functional lens 222 is even closer to the user's eyes. In FIG. 3C, the inner end 221b of the connecting device 221 connects to the functional lens 222 by a buckled element 223 so that the functional lens 222 is even closer to the user's eyes. It should be noted that the ways in which the connecting device 221 and the functional lens 222 are connected are only provided for purposes of illustration but not limitation.

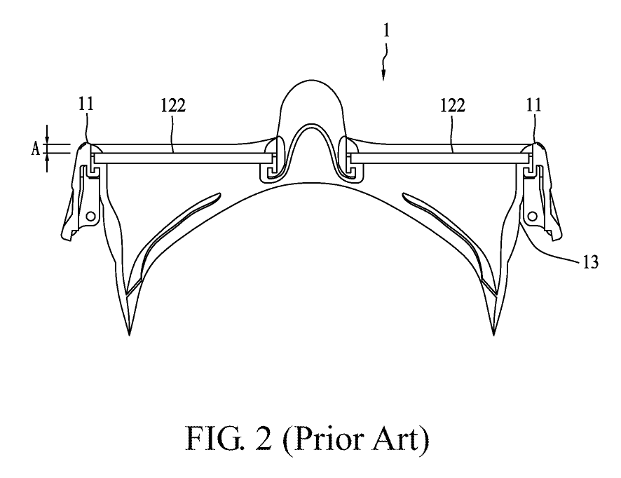

As can be seen by comparing the conventional swimming goggle structure 1 shown in FIG. 2 with the swimming goggle structure 2 of the present invention shown in FIG. 4, the lens 122 of the conventional swimming goggle structure 1 is restrained in outline by the mask 13, so that the distance A between the lens 122 and outer edge of the frame assembly 11 is relatively small. Consequently, the lens 122 of the conventional swimming goggle structure 1 has a large distance from the user's eyes, which requires the use of a lens 122 with a large area to provide a normal field of vision. On the other hand, as shown in FIG. 4, by means of the connecting device 221, the functional lens 222 of the present invention is no longer restrained in outline by the mask, so the distance between the functional lens 222 and an outer edge of the frame assembly 21 (the distance B shown in FIG. 4) can be increased; that is, the distance B is greater than the distance A. As a result, the functional lens 222 of the swimming goggle structure 2 of the present invention has a shorter distance from the user's eyes, which allows for using a functional lens 222 that has a smaller area to obtain the same normal field of vision as the conventional lens with a larger area. Thereby, the problem of tunnel vision is obviated.

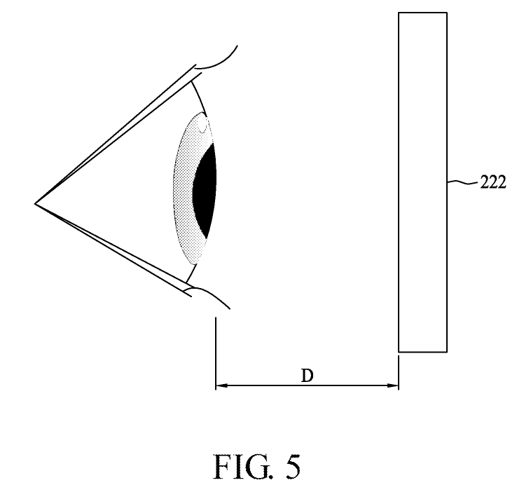

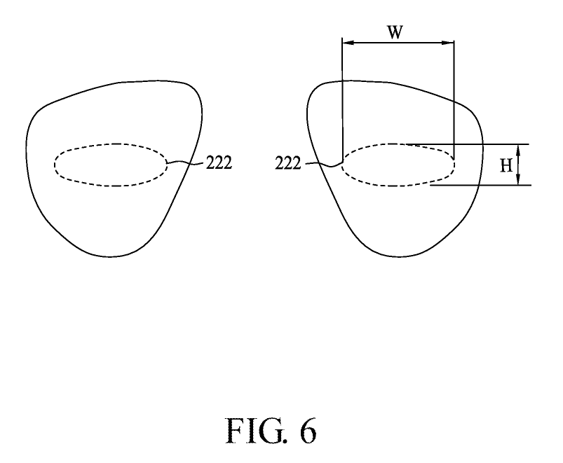

In reference to FIGS. 5 and 6, examples of the present invention are illustrated therein. As an example, FIG. 5 shows that the distance D between the user's eyes and the functional lens 222 ranges substantially between 4 mm and 16 mm. Next, in reference to FIG. 6, as described above, the functional lens 222 can be remarkably reduced in area than the conventional ones; for example, the functional lens 222 may have the width range W substantially reduced to 32˜50 mm, and the height range H substantially reduced to 12˜50 mm. Accordingly, less time and labor and lower cost will be needed to produce the functional lens.

Furthermore, because the connecting device 221 is made of a transparent material, such as tempered glass, transparent silica gel, transparent plastics or transparent acrylics, and covers the functional lens 222 along an edge of the functional lens 222, the connecting device 221 may function like a cushioning material to strengthen the shock absorption capability, increase the shock resistance and reduce the overall weight of the swimming goggle structure 2, thereby making the user feel more comfortable during use.

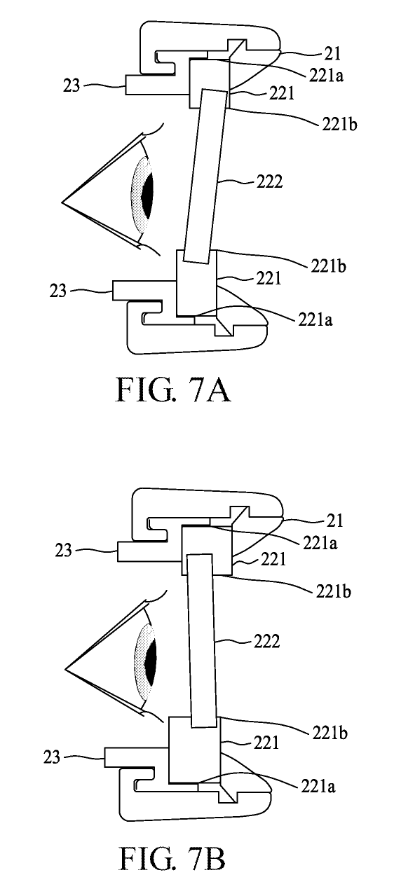

As shown in FIG. 7A, the lens assembly 22 of the present invention may further have the lens inclined by utilizing the connecting relationship between the connecting device 221 and the functional lens 222. In particular, with the corresponding arrangement of the connecting device 221, an angle of 0 to +90° as shown in FIG. 7A or an angle of 0 to −90° as shown in FIG. 7B may be included between the functional lens 222 and a vertical line depending on different needs. In this way, suitable swimming goggles may be chosen by the user for use in different environments to obtain a large-angle downward or upward field of view in underwater sports.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A swimming goggle structure, comprising:
a frame assembly, having a slot disposed on a rim of the frame assembly and defining a receiving space; and
a lens assembly, comprising a connecting device and a functional lens, the connecting device extending toward a user's eyes, the connecting device having an outer end and an inner end, the outer end fit in the slot so that the lens assembly is adapted to be embedded in the receiving space of the frame assembly, and the inner end connecting to the functional lens.

2. The swimming goggle structure as claimed in claim 1, wherein the connecting device comprises a transparent material.

3. The swimming goggle structure as claimed in claim 2, wherein the transparent material comprises transparent plastic.

4. The swimming goggle structure as claimed in claim 2, wherein the transparent material comprises transparent silica gel.

5. The swimming goggle structure as claimed in claim 2, wherein the transparent material comprises transparent acrylics.

6. The swimming goggle structure as claimed in claim 1, wherein the inner end of the connecting device connects to the functional lens by an adhesive.

7. The swimming goggle structure as claimed in claim 1, wherein the inner end of the connecting device connects to the functional lens by a buckled element.

8. The swimming goggle structure as claimed in claim 1, wherein the inner end of the connecting device connects to the functional lens by an injection molding method.

9. The swimming goggle structure as claimed in claim 1, wherein the outer end of the connecting device connects to the frame assembly by an adhesive.

10. The swimming goggle structure as claimed in claim 1, wherein the outer end of the connecting device connects to the frame assembly by a buckled element.

11. The swimming goggle structure as claimed in claim 1, wherein the outer end of the connecting device connects to the frame assembly by an injection molding method.

12. The swimming goggle structure as claimed in claim 1, wherein a distance between the outer end of the connecting device and the user's eyes is greater than a distance between the inner end and the user's eyes.

13. The swimming goggle structure as claimed in claim 12, the distance between the inner end and the user's eyes is between 4 mm to 16 mm.

14. The swimming goggle structure as claimed in claim 1, wherein a width of the functional lens is between 32 mm to 50 mm, and a height of the functional lens is between 12 mm to 50 mm.

15. The swimming goggle structure as claimed in claim 1, wherein an angle between the functional lens and a vertical line is between 0 to 90 degrees.

16. The swimming goggle structure as claimed in claim 1, wherein an angle between functional lens and a vertical line is between 0 to −90 degrees.

17. The swimming goggle structure as claimed in claim 1, wherein the inner end of the connecting device connects to the functional lens, the outer end of the connecting device is received in the slot and connects to the frame assembly so that the lens assembly is formed integrally.
